(19) United States
(12) Patent Application Publication
(10) Pub. No.: US 2020/0096787 A1
(43) Pub. Date: Mar. 26, 2020

(54) OPHTHALMIC COMMUNICATION WITH USER METHOD AND SYSTEM

(71) Applicant: Johnson & Johnson Vision Care, Inc., Jacksonville, FL (US)

(72) Inventors: Scott Humphreys, Greensboro, NC (US); Donald K. Whitney, Melbourne, FL (US); Adam Toner, Jacksonville, FL (US)

(21) Appl. No.: 16/138,537

(22) Filed: Sep. 21, 2018

(51) Int. Cl. G02C 7/04; A61F 2/16

(52) U.S. Cl. CPC G02C 7/041; A61F 2250/0002; A61F 2/1624

(57) ABSTRACT

Methods and systems for communication with a user of an ophthalmic device are disclosed. An example method may comprise determining, by a processor disposed on or in an ophthalmic device, an occurrence of an event, wherein the ophthalmic device is disposed within or upon an eye of a user and comprises an ophthalmic lens and a variable-optic element, and wherein the variable-optic element is configured to change a refractive power of the ophthalmic lens; causing the ophthalmic lens to adjust from a first refractive power to a second refractive power indicative of the occurrence of the event; determining that a time threshold is satisfied; and causing, in response to determining that the time threshold is satisfied, the ophthalmic lens to adjust the ophthalmic lens from the second refractive power to the first refractive power.

### BACKGROUND

The present invention relates to ophthalmic devices, such as wearable lenses, including contact lenses, implantable lenses, including intraocular lenses (IDLs) having embedded lens controlling elements, and more specifically, to use the embedded lens controlling elements to communicate with a user.

Near and far vision needs exist for all. In young non-presbyopic patients, the normal human crystalline lens has the ability to accommodate both near and far vision needs and those viewing items are in focus. As one ages, the vision is compromised due to a decreasing ability to accommodate as one ages. This is called presbyopia.

The use of Adaptive optics/powered lens products are positioned to address this and restore the ability to see items in focus. But what is required is knowing when to activate or actuate the optical power change. While a manual indication or use of a key fob to signal when a power change is required is one way to accomplish this change. However, leveraging anatomical/biological conditions/signals may be more responsive, more user friendly and potentially more “natural” and thus more pleasant.

A number of things happen when we change our gaze from far to near. Our pupil size changes, our line of sight from each eye converge in the nasal direction coupled with a somewhat downward component as well. However, to sense/measure these items are difficult, one also needs to filter out certain other conditions or noise, (e.g.: blinking, what to do when one is lying down, or head movements).

In reference to FIG. 4, when observing an object in each eye the visual axis points toward the object or Target. Since the two eyes are spaced apart (distance b) and the focal point is in front, a triangle is formed. Forming a triangle allows the relationship of rotation angles for the left and right eyes (θL and θR, respectively) of each visual axis to the distance (Y) the object is from the eyes to be determined. Since the distance (Y) is what determines if a change in optical power is required, then the relationship between angles and the distance between the eyes and allows a system to make a decision regarding when to change the optical power. Note that the sign of the angles may be such that a counter clockwise rotation is represented by a positive angle value, and a clockwise rotation is represented by a negative angle value. Thus for normal conditions the left angle is between zero and a negative value, and the right angle is between zero and a positive value. The difference between the right and left angles may be referred to as a vergence angle.

Sensing of multiple items may be required to remove/mitigate any false positive conditions that would indicate a power change is required when that is not the case. Use of an algorithm may be helpful. Additionally, near or far distance threshold levels for determining when to use near or far focus, respectively, may vary from patient (user) to patient (user), thus some form of calibration will likely be required as well. Additionally, it may be desirable to communicate with the user during calibration or other operations. For example, feedback, acknowledgements, and other information may be communicated to a user via an ophthalmic device.

### SUMMARY

According to one aspect of the present invention, an example ophthalmic system comprising: an ophthalmic device configured to be disposed within or upon an eye of a user and comprising an ophthalmic lens and a variable-optic element, the variable-optic element being configured to change a refractive power of the ophthalmic lens; and a processor disposed in or on the ophthalmic device, wherein the processor is configured for: determining occurrence of an event; causing the ophthalmic lens to adjust from a first refractive power to a second refractive power indicative of the occurrence of the event; determining that a time threshold is satisfied; and causing, in response to determining that the time threshold is satisfied, the ophthalmic lens to adjust the ophthalmic lens from the second refractive power to the first refractive power.

According to another aspect of the present invention, another example ophthalmic system comprising: an ophthalmic device configured to be disposed within or upon an eye of a user and comprising an ophthalmic lens and a variable-optic element, the variable-optic element being configured to change a refractive power of the ophthalmic lens; and a processor disposed in or on the ophthalmic device, wherein the processor is configured for: receiving data; causing modification of an accommodation setting based on the data; and causing a return to the accommodation setting after a predefined time period.

According to another aspect of the present invention, another example ophthalmic system comprising: an ophthalmic device configured to be disposed within or upon an eye of a user and comprising an ophthalmic lens and a variable-optic element, the variable-optic element being configured to change a refractive power of the ophthalmic lens; and a processor disposed in or on the ophthalmic device, wherein the processor is configured for: determining to communicate with the user via the ophthalmic device; determining, based on determining to communicate with the user, a first accommodation parameter; causing, based on the first accommodation parameter, the ophthalmic device to adjust the ophthalmic lens from a first focal length to a second focal length; and causing, in response to a time threshold being satisfied, the ophthalmic device to adjust, based on a second accommodation parameter, the ophthalmic lens from the second focal length to the first focal length.

According to another aspect of the present invention, a method comprising determining, by a processor disposed on or in an ophthalmic device, an occurrence of an event, wherein the ophthalmic device is disposed within or upon an eye of a user and comprises an ophthalmic lens and a variable-optic element, and wherein the variable-optic element is configured to change a refractive power of the ophthalmic lens; causing the ophthalmic lens to adjust from a first refractive power to a second refractive power indicative of the occurrence of the event; determining that a time threshold is satisfied; and causing, in response to determining that the time threshold is satisfied, the ophthalmic lens to adjust the ophthalmic lens from the second refractive power to the first refractive power.

According to another aspect of the present invention, another example method comprising receiving, by a processor disposed on or in an ophthalmic device, data, wherein the ophthalmic device is disposed within or upon an eye of a user and comprises an ophthalmic lens and a variable-optic element, and wherein the variable-optic element is configured to change a refractive power of the ophthalmic lens; causing modification of an accommodation setting based on the data; and causing a return to the accommodation setting after a predefined time period.

According to another aspect of the present invention, another example method comprising determining, by a processor disposed on or in an ophthalmic device, to communicate with a user via the ophthalmic device, wherein the ophthalmic device is disposed within or upon an eye of the user and comprises an ophthalmic lens and a variable-optic element, and wherein the variable-optic element is configured to change a refractive power of the ophthalmic lens; determining, based on determining to communicate with the user, a first accommodation parameter; causing, based on the first accommodation parameter, the ophthalmic device to adjust the ophthalmic lens from a first focal length to a second focal length; and causing, in response to a time threshold being satisfied, the ophthalmic device to adjust, based on a second accommodation parameter, the ophthalmic lens from the second focal length to the first focal length.

### BRIEF DESCRIPTION OF THE OF THE DRAWINGS

FIG. 1 shows an exemplary implementation according to an embodiment of the present invention.

FIG. 2 shows a flowchart according to an embodiment of the present invention.

FIG. 3 shows another exemplary implementation according to an embodiment of the present invention.

FIG. 4 shows an example of focus determination.

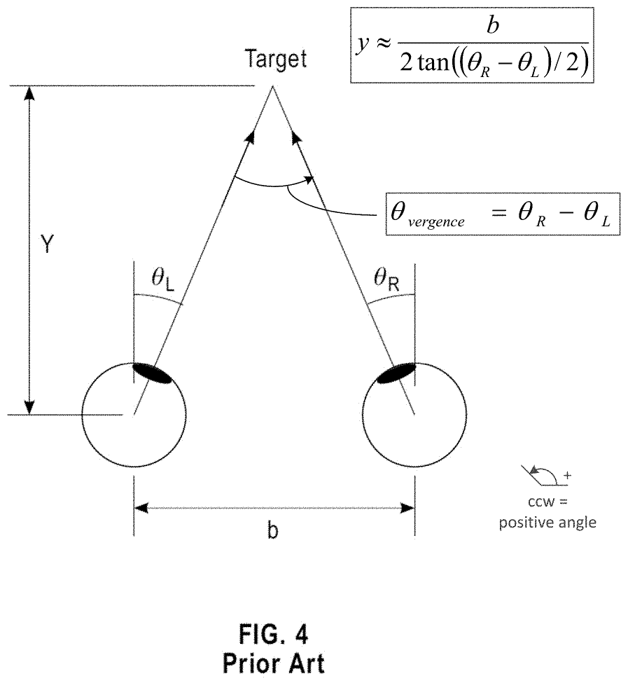

FIG. 5 shows another flowchart according to an embodiment of the present invention.

FIG. 6 shows another flowchart according to an embodiment of the present invention.

FIG. 7 shows another flowchart according to an embodiment of the present invention.

FIG. 8 shows another flowchart according to an embodiment of the present invention.

FIG. 9 shows an example device according to an embodiment of the present invention.

FIG. 10 is a diagram illustration regions of an ophthalmic device.

### DETAILED DESCRIPTION

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting. As will be appreciated by one skilled in the art, aspects of the present invention may be embodied as a system, method or computer program product.

Throughout the specification the terms ophthalmic device and ophthalmic device are utilized. In general terms, an ophthalmic device may include contact lenses, intraocular lenses, spectacle lenses and punctal plugs. However, in accordance with the present disclosure, an ophthalmic device is one for eye disease treatment, vision correction and/or enhancement and preferably includes at least one of punctal plugs, spectacle lenses, contact lenses and intraocular lenses. An intraocular lens or IOL is a lens that is implanted in the eye and replaces the crystalline lens. It may be utilized for individuals with cataracts or simply to treat various refractive errors. An IOL typically comprises a small plastic lens with plastic side struts called haptics to hold the lens in position within the capsular bag in the eye. Any of the electronics and/or components described herein may be incorporated into IOLs in a manner similar to that of contact lenses. A punctal plug or occluder is an ophthalmic device for insertion into a punctum of an eye in order to treat one or more disease states. While the present disclosure may be utilized in any of these devices, in preferred exemplary embodiments, the present disclosure is utilized in contact lenses or intraocular lenses.

The present disclosure may be employed in a powered ophthalmic device comprising an electronic system, which actuates a variable-focus optic or any other device or devices configured to implement any number of numerous functions that may be performed.

Because everyone's eyes are a bit different, (eg: pupil spacing and location, lens-on-eye position, etc.), even at a fixed close distance, the difference in gaze angles formed by each eye relative to the head when observing an object, or vergence angle, differs from person to person. It may be important once lenses are placed on the eye to measure or calibrate an initial vergence angle or to form an estimate of an initial vergence angle, so that changes in the vergence angle can be assessed while in service. This value can be used for subsequent calibration calculations. During calibration, or otherwise, feedback and other notifications may be provided to the user to indicate updated settings, failure or completion of an operation, and other information.

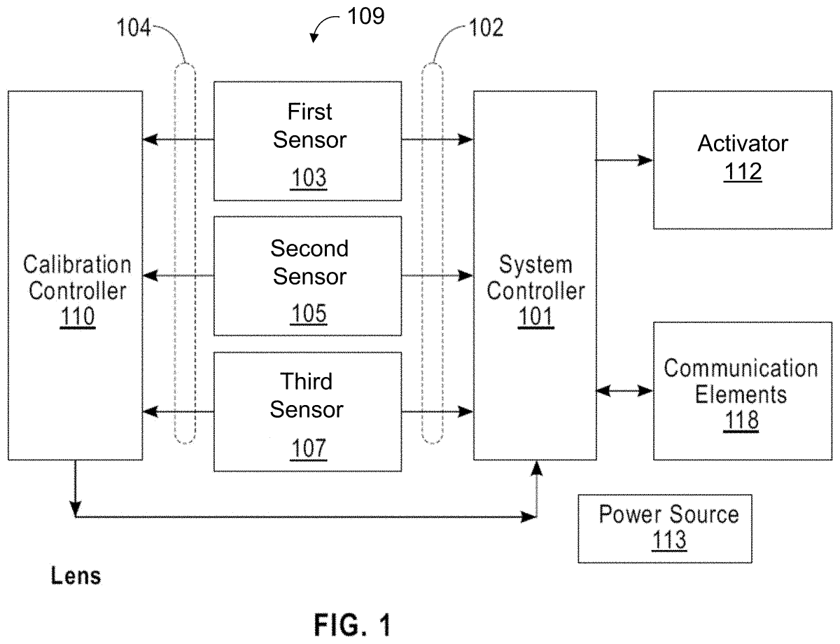

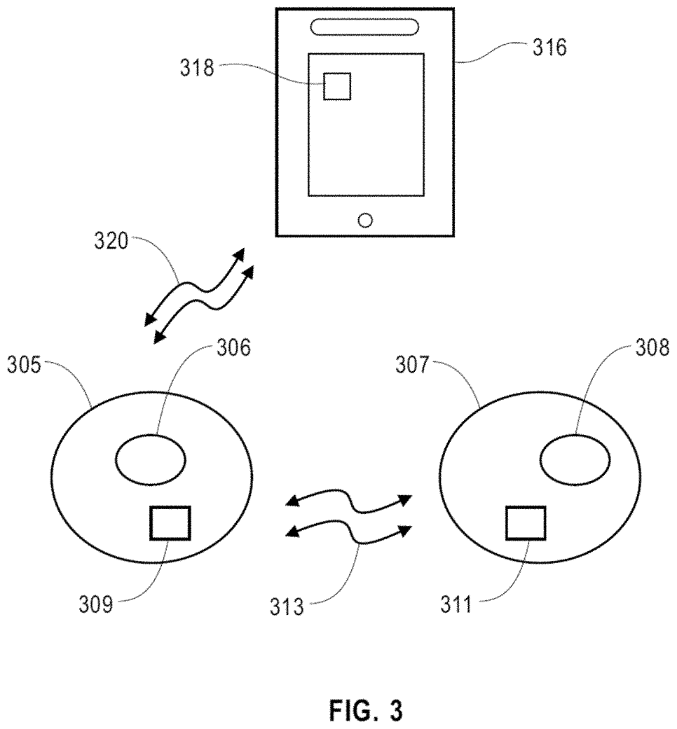

Now referring to FIG. 1, an exemplary implementation shows a system according to an embodiment of the present invention. The system can be disposed in or on an ophthalmic device. The ophthalmic device can comprise a contact lens or an implantable lens, or a combination of both. The ophthalmic device can be configured to be disposed within or upon an eye of a user. The contact lens comprises a soft or hybrid contact lens. The ophthalmic device can be part of system of at least two ophthalmic devices, as shown in FIG. 3.

A system controller 101 controls a lens activator 112 than changes the adaptive optics/powered lens (see FIG. 3) to control the ability to see both near and far items in focus. The system controller 101 can comprise a processor, memory, and/or the like. The system controller 101 may be a microcontroller or a digital hardware controller comprising state machines. The system controller 101 (e.g., the processor) can be operably coupled to a sensor element 109. The system controller 101 receives signals 102 (e.g., data signals, control signals) from the sensor element 109.

The sensor element 109 can comprise a plurality of sensors (103, 105 and 107). Examples of sensors can comprise a multidimensional sensor, a capacitive sensor, an impedance sensor, an accelerometer, a temperature sensor, a displacement sensor, a neuromuscular sensor, an electromyography sensor, a magnetomyography sensor, a phonomyography, or a combination thereof. The plurality of sensors (103, 105 and 107) can comprise a lid position sensor, a blink detection sensor, a gaze sensor, a divergence level sensor, an accommodation level sensor, a light sensor, a body chemistry sensor, neuromuscular sensor, or a combination thereof. The plurality of sensors (103, 105 and 107) can comprise one or more contacts configured to make direct contact with tear film of an eye of the user.

As an illustration, the plurality of sensors (103, 105 and 107) can comprise a first sensor 103, such as a first multidimensional sensor that includes an X-axis accelerometer. The plurality of sensors (103, 105 and 107) can comprise a second sensor 105, such as a second multidimensional sensor that includes a Y-axis accelerometer. The plurality of sensors (103, 105 and 107) can comprise a third sensor 107, such as a third multidimensional sensor that includes a Z-axis accelerometer. As another embodiment, the three axis accelerometers can be replaced by a three-axis magnetometer. In some embodiments the plurality of sensors 103, 105 and 107 may be configured as a 3-axis accelerometer.”

Calibration would be similar because each axis would potentially require calibration at each extreme of each axis. The plurality of sensors (103, 105 and 107) further provide calibration signals 104 to a calibration controller 110. The calibration controller 110 conducts a calibration sequence based on the calibration signals from the plurality of sensors (103, 105 and 107) as a result of user actions which is sensed by the plurality of sensors (103, 105 and 107) and provides calibration control signals 102 to the system controller 101. The system controller 101 further receives from and supplies signals to communication elements 118. Communication elements 118 allow for communications between user lens and other devices such a near-by smartphone. A power source 113 supplies power to all of the above system elements. The power source can comprise a battery. The power sources may be either a fixed power supply, wireless charging system, or may be comprised of rechargeable power supply elements. Further functionality of the above embedded elements is described herein. As another embodiment, the three axis accelerometers can be replaced by a three-axis magnetometer. Calibration would be similar because each axis would potentially require calibration at each extreme of each axis.

The system controller 101 can be configured to perform a communication operation. The communication operation may be any operation that communicates feedback or other information to a user. The communication operation may be performed by a single ophthalmic device or multiple ophthalmic devices. For example, the system can comprise at least two ophthalmic devices, as shown later in FIG. 3. For purposes of illustration multiple ophthalmic devices are described, one or more (or each) of which can be an ophthalmic device as shown in FIG. 1. For example, a first ophthalmic device can be configured to be disposed within or upon a first eye of a user. As illustrated in FIG. 1, the first ophthalmic device can comprise a first sensor system. The first sensor system can comprise a first sensor. The first ophthalmic device may comprise a first processor. The first processor may be operably connected to the first sensor. A second ophthalmic device can be configured to be disposed within or upon a second eye of the user. The second ophthalmic device can comprise a second sensor system. The second sensor system can comprise a second sensor. The second ophthalmic device may comprise a second processor. The second processor may be configured to be operably connected to the second sensor.

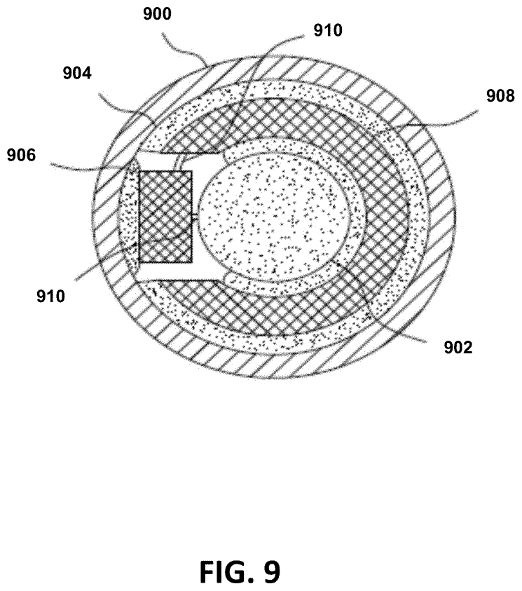

The first ophthalmic device may comprise a first ophthalmic lens. The first ophthalmic device may comprise a first variable-optic element. The first variable-optic element may be configured to change a parameter (e.g., change a value of the parameter) of the first ophthalmic lens. The parameter may comprise a refractive power, a focal length, an opacity, a polarization, a focal path, an optical power, a focal length, a refractive index, gradient of the refractive index, and/or the like of the ophthalmic lens. The first ophthalmic lens may have a first optic zone and a first peripheral zone. The first variable-optic element may be incorporated into the first optic zone. The first processor may be incorporated into the first peripheral zone. The second ophthalmic device may comprise a second ophthalmic lens. The second ophthalmic device may comprise a second variable-optic element. The second variable-optic element may be configured to change a refractive power of the second ophthalmic lens. The second ophthalmic lens may have a second optic zone and a second peripheral zone. The second variable-optic element may be incorporated into second first optic zone. The second processor may be incorporated into the second peripheral zone. An example ophthalmic lens having an optic zone and a peripheral zone are shown in FIG. 9 and described in further detail in the accompanying description.

In an aspect, the communication operation may be any operation to communicate with a user using an ophthalmic device. An ophthalmic device may store a plurality of communications operations associated with corresponding events. If an event is detected, the ophthalmic device may determine an associated communication operation and perform the communication operation to communicate with the user.

An example communication operation may comprise a single operation or a sequence of operations. The communication operation may provide feedback (e.g., acknowledgement) to the user regarding an operation performed by the ophthalmic device, such as performing a calibration, changing a setting, reaching a power level, and/or the like. Various patterns of activation of a lens or other element (e.g., light haptic driver) could be used to communicate to the user to a status of the ophthalmic device (e.g., or lens thereof). The communication operation may comprise a flicker operation, activation (e.g., turning on) of a lens, deactivation of a lens, toggling the lens between activated and deactivated states, activating a haptic driver, blinking a light, projecting an image on the user's retina, a combination thereof, and/or the like.

As an example, a flicker operation may comprise changing a parameter (e.g., an accommodation parameter, a focal length, a refractive power) associated with the ophthalmic lens for a limited time period. The parameter and/or the time period may be selected to minimize disruption to the user (e.g., cause less than a threshold amount of disruption). Following the time period, the ophthalmic lens may be returned to the original setting, or changed to a new setting. Different types time periods may be associated with communicating different information to a user. For example, if calibration (e.g., or a step thereof) fails, then a first time period may be used. If calibration (e.g., or a step thereof) is successful, then a second time period may be used. The second time period may be longer or shorter than the first time period (e.g., such that the user can distinguish between the two). Different numbers of flicker events may be associated with communicating different information to a user. For example, if calibration (e.g., or a step thereof) fails, then a first number may be used. If calibration (e.g., or a step thereof) is successful, then a second number may be used. The second number may be more or less than the first number (e.g., such that the user can distinguish between the two).

The communication operation may comprise changing a parameter based on polarity. For example, a user may have two ophthalmic devices (e.g., one for each eye). A first polarity may be assigned to a first ophthalmic device, and a second polarity may be assigned to a second ophthalmic device. The communication operation may comprise performing the flicker operation (e.g., or any other communication described herein) on one of the ophthalmic devices (e.g., the one associated with a particular polarity indicated by the communication operation). The communication operation may comprise alternating between performing the flicker operation (e.g., or any other communication described herein) for the first ophthalmic device and performing the flicker operation for the second ophthalmic device. The communication operation may comprise a sequence of communication operations, some of which are performed based on the first polarity and others of which are performed based on the second polarity. The specific ophthalmic device use to perform the communication operation may be based on a polarity associated with event and/or the communication operation.

The communication operation may comprise modifying (e.g., activating, deactivating, changing a value of) one or more regions of the ophthalmic device. The one or more regions may be a part of an ophthalmic lens or attached to an ophthalmic lens. For example, the regions may comprise one or more concentric rings, circles, squares, and/or any other appropriate shape. A current may be applied to one or more of the regions (e.g., concentric rings) to modify an appearance of the regions (e.g., concentric rings) and/or modify how light passes through the regions. For example, modifying a region (e.g., concentric ring) may comprise increasing or decreasing an opacity of the region (e.g., concentric ring). Modifying a region (e.g., concentric ring) may comprise generating light from the region (e.g., concentric ring). The region (e.g., concentric ring) may be darkened to generate a dark (e.g., or blackened) line, ring, square, circle, or any other shape. The region (e.g., concentric ring) may be modified based on a pattern. For example, a single region (e.g., concentric ring) may be modified a number of times and a length of time based on the pattern. Multiple regions (e.g., concentric ring) may be modified in a sequence. The opacity and/or pattern may be selected based on the event, a category of the event (e.g., importance level, time of occurrence), and/or the like.

Modifying one or more regions of the ophthalmic device may comprise changing a refractive index of all or a portion of the ophthalmic device. The refractive index may be changed by applying a current, voltage, and/or an electric field to all or a portion of the ophthalmic lens. Modifying one or more regions of the ophthalmic device may comprise modifying a gradient of the refractive index. Different regions may have the refractive index changed by different amounts in order to create a gradient effect. The refractive index may be changed to change a focal path.

Modifying one or more regions of the ophthalmic device may comprise modifying (e.g., activating, deactivating, changing a value of) an optic zone. Modifying the optic zone may comprise changing an aperture. The aperture may be decreased or increased in size. Changing the aperture may change a focal length, thereby resulting in causing vision to become blurred or focused. Modifying the optic zone may comprise modifying a state of one or more lenslets. The one or more lenslets may disposed be in the optic zone or the peripheral zone of the ophthalmic lens. The one or more lenslets may be centered in the optic zone. An example lenslet may be smaller than a pupil of the eye. The lenslet may comprise a liquid meniscus.

The communication operation may comprise modifying a state of a liquid meniscus region. The liquid meniscus region may be within a lenslet, within the optic zone, and/or the like. The liquid meniscus region may comprise a liquid containing cell for retaining a volume of two or more liquids. A lower surface, which may be non-planar. The depression or recess may be conical, cylindrical, and/or the like. The depression or recess may comprise a drop of a first liquid. The first liquid may comprise an insulating liquid. A remainder of the cell (e.g., depression or recess) may comprise a second liquid. The second liquid may comprise an electrically conductive liquid. The second liquid may be non-miscible with the first liquid. The second liquid may have a different refractive index than the first liquid. The second liquid may have a similar or same density as the first liquid. An annular electrode (e.g., which may be open facing a recess) may be positioned on the rear face of a lower plate. Another electrode may be placed in contact with the conductive liquid. Application of a voltage across the electrodes is utilized to modify the curvature of the interface between the two liquids, according to the voltage (V) applied between the electrodes. A beam of light passing through the cell normal to the upper plate and the lower plate and in the region of the drop will be focused to a greater or lesser extent according to the voltage applied to the electrodes. The conductive liquid is typically an aqueous liquid, and the insulating liquid is typically an oily liquid. Generally, the system controller 101 controls the application of voltage across the electrodes of a liquid meniscus region and thereby controls the optical characteristics of the ophthalmic lens. The system controller 101 may also monitor and track variables related to the liquid meniscus region, such as for example, a current state of optical characteristics. The liquid meniscus region may be toggled between multiple states according to any pattern to in order to communicate with the user. For example, the voltage may be switched between two voltages to switch the liquid meniscus region between two states. The difference in the voltages may be determined based on a category of the event, category of the communication, and/or an importance level, and/or the like. For example, a greater change between states may be caused for a greater level of importance.

Modifying one or more regions of the ophthalmic device may comprise modifying a polarization of one or more regions. A current, voltage, electrical field, and/or the like may be applied to one or more regions thereby changing a polarization of the region. For example, the region may be modified from no polarization to either polarized state. The region may be modified from one polarized state to another. The result of the modifying may be a darkening effect (e.g., by blocking light of the opposite polarization). The polarization may be changed in any pattern described herein. For example, the polarization may be changed as part of a flicker operation.

An event associated with a communication operation may comprise beginning a step in a calibration operation, completing a step in a calibration operation, beginning a calibration operation, completing a calibration operation, and/or the like. The event may comprise modifying a system parameter, such as a vergence setting, an accommodation parameter, a customized accommodation threshold, and/or the like. The event may comprise one or more of receipt of a user instruction, receipt of a notification from a remote device, or a condition is triggered. As an example, an ophthalmic device may detect that a user is receiving a call or a message on a mobile device. The ophthalmic device may perform a communication, such as a flicker operation, to notify the user regarding the message or call. An example condition that may be triggered can comprise a power level of the ophthalmic device falling below a threshold, an error state being detected (e.g., memory error, processing error, heat level), a health warning (e.g., user temperature, eye condition, eye lubrication level, blood sugar level), and/or the like.

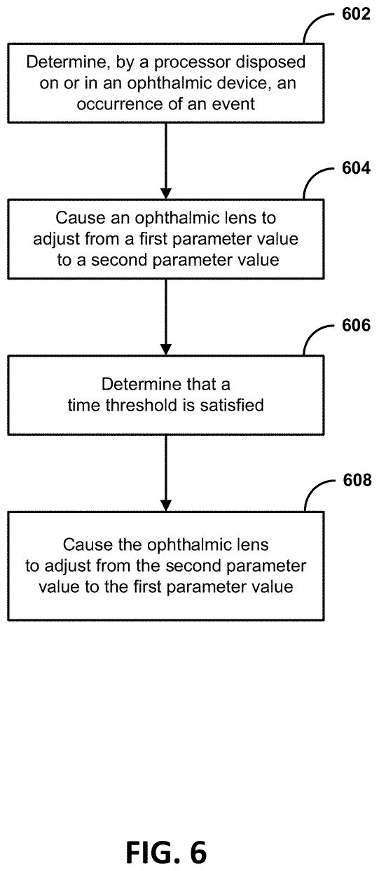

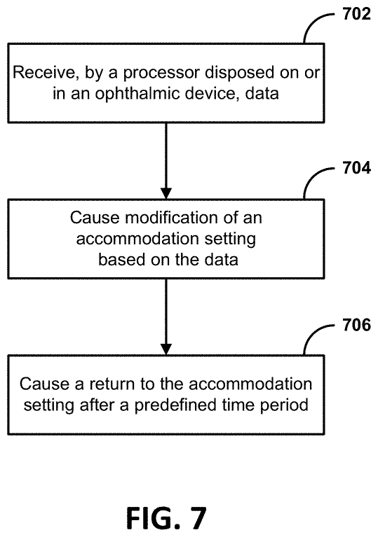

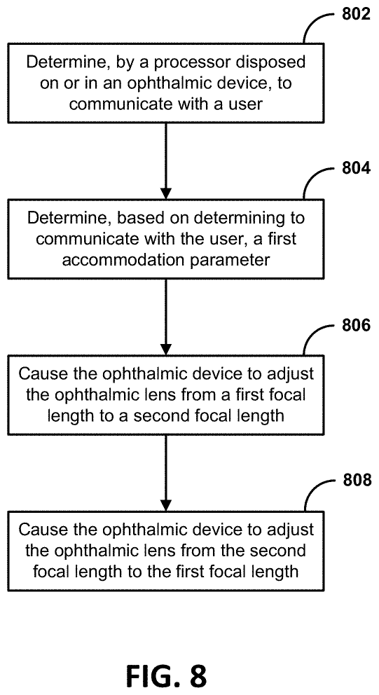

Example communication operations are described further throughout the specification. For example, the communication operation may be performed as described in any combination of steps illustrated in FIG. 6, FIG. 7, and FIG. 8. Specific examples are provided in the context of calibration as well as other contexts described herein.

In the context of using sensors, specifically accelerometers, to determine vergence, there are opportunities to calibrate. Offsets, due to the micro-electromechanical systems (MEMS) and/or due to the electronics, mounting on the interposer, etc. can cause variations with the algorithms and thus cause some errors in the measurement of vergence. In addition, human anatomy from person to person is different. For instance, eye to eye space can vary from 50 to 70 mm and this variation alone can cause a change in trigger or threshold angles. So there is a need to take some of these variables out of the measurement, thus calibration and customization performed by the current embodiment when the lens are on the user. This serves to improve the user experience by both adding the preferences of the user and to reduce the effects of the above-mentioned variations.

The sensors (103, 105 and 107) measure acceleration both from quick movements and from gravity (9.81 m/s²). Accelerometers usually produce a code that is in units of gravity (g), thus the determination of vergence depends on the measurement of gravity to determine position, but other methods may depend on the acceleration of the eye. There are going to be differences and inaccuracies that will require base calibration before use calibration.

The current embodiment uses three sensors on each lens. However, calibration may be done using two sensors, e.g., the X-axis accelerometer 103 and the Y-axis accelerometer 105. In either embodiment, each accelerometer has a full scale plus, full scale minus, and zero position. The errors could be offset, linearity, and slope errors. A full calibration would calibrate to correct all three error sources for all of axes sensors being used.

One way to calibrate the sensors is to move them such that each axis is completely perpendicular with gravity, thus reading 1 g. Then the sensor would be turned 180 degrees and it should read −1 g. From two points, the slope and intercept can be calculated and used to calibrate. This is repeated for the other two sensors. This is an exhaustive way of calibrating the sensors and thus calibrating the vergence detection system.

Another way is to reduce the calibrate effort for the lens is to have the wearer do just one or two steps. One way is to have the wearer look forward, parallel to the floor, at a distance wall. Measurements taken at this time can be used to determine the offset of each axis. Determining the offset for each axis in the area where the user will spend most of the time provides a greater benefit to maintain accuracy. The ophthalmic device may indicate that the offset has been determined by performing a communication operation as described further herein.

Given that everyone is a little different, customizable features can prove a better user experience for all users than a one size fits all approach. When using the lens with just two modes, accommodation (near focus) and gaze (far focus), then the point where there is a switch from gaze to accommodation can depend on several parameters that affect the user experience in addition to a vergence or distance threshold.

The threshold at which to switch from gaze to accommodation depends on the user, the user's eye condition, the magnification of the lens, and the tasks. For reading, the distance between the eye and book is about 30 cm, while computer usage is about 50 cm. A threshold set for 30 cm wouldn't work well for computer work, but 50 cm could work for both. However, this longer threshold could be problematic for other tasks by activating too early, depending on the magnification and the user's own eye condition. Thus, the ability to alter this threshold, both when the lens is first inserted and at any time afterwards as different circumstances could require different threshold points, provides the user customization to improve visibility, comfort and possibly safety. Even having several present thresholds is possible and practical, where the user would choose using the interfaces described here to select a different threshold. In addition, the user could alter the threshold or other parameters by re-calibrating per the embodiments of the present invention as described hereafter. When a user has altered the threshold, the ophthalmic device may indicate that the threshold has been altered by performing a communication operation as described further herein.

Still referring to FIG. 1, switching from gaze to accommodation, the system uses the threshold as the activation point. However, going from accommodation to gaze the threshold is shifted to a greater distance, which is called hysteresis and the difference between the thresholds the amount of hysteresis. Hysteresis is added to prevent uncertainty when the user is viewing at a distance close to the threshold and there are small head movements or eye movements which may cause a lens without hysteresis to switch from gaze to accommodation to gaze, etc. The threshold and the amount of hysteresis could be set such that in most cases the lens switches to the desired focus setting without further effort from the user beyond their natural way of looking at near or far objects. If the threshold is set too close, then the user may need to bring an object closer than they would like in order for the lens to change focus. Then they could move the object further away, within the amount of hysteresis, and the lens will remain in the accommodation (near focus) setting. Similarly for an object in at a middle distance, the user may need to look at a farther distance beyond the far threshold (that is, beyond the threshold distance plus the amount of hysteresis) for the lens to change to the gaze (far focus) setting. Then the user may again view the object and the lens will remain in the far focus as long as the user does not bring the object closer than the threshold. The threshold and/or the amount of hysteresis can be set or adjusted in several ways: one, the doctor fitting the lenses can change the values, two, the user can change the values via a lens interface, and three, an adaptive algorithm can adjust the values based on the habits of the user. The ophthalmic device may indicate that the threshold or the amount of hysteresis have been changed by performing a communication operation as described further herein.

Custom Modes are common now in cars, e.g. sport, economy, etc. which allow the user to pick a mode based on anticipated activity where the system alters key parameters to provide the best experience. Custom Modes also may be integrated into the lens of some embodiments. Calibration and customization settings can be optimized for a given mode of operation. If the user is working in an office, it is likely that the user will need to switch between states (gaze and accommodation) often, or even to switch between using two different vergence angle or distance thresholds because of the nature of different tasks. Changes in the threshold, hysteresis, noise immunity, and possible head positions could be made to provide quicker transitions, possible intermediate vergence positions, and optimization for computer tasks, as well as, tasks that there is a lot if switching between gaze and accommodation. Thus, options to switch the lens into different modes to optimize the lens operation can provide an enhanced user experience. Furthermore, in an “Exercise” mode, the noise filtering may be increased to prevent false triggering and additional duration of positive signal may be required before switching to prevent false switching of the lens being triggered by stray glances while running. A “Driving” mode might configure the lens for distant use or to operate in a “manual” mode only. Of course, various other modes could be derived as part of the embodiments of the present invention. The ophthalmic device may indicate that the user mode has been changed by performing a communication operation as described further herein. For example, when a driving mode is activated, the ophthalmic device may perform the flicker operation to indicate the change. The flicker operation may be associated with the driving mode and configured to minimize disruption of driving.

In today's world, the smartphone is becoming a person's personal communications device, library, payment device, and general connection to the world. Apps for the smartphone cover many areas and are widely used. One possible way to interact with the lens of the present invention is to use a phone application, or “app.” The app could provide ease of use where written language instructions are provided and the user can interact with the app providing clear instructions, information, and feedback. Voice activation options may also be included. For instance, the app may provide prompts for sensor calibrations by instructing the user to look forward and prompting the user to acknowledge the process start. The app could provide feedback to the user to improve the calibration and instruct the user what to do if the calibration is not accurate enough for optimal operation. This would enhance the user experience.

Additional indicators may be simple responses from the system to indicate start of a calibration cycle, successful completion, and unsuccessful completion. Methods to indicate operation include, but not limited to, blinking lights, vibrating haptics drivers, and activating the lens. Various patterns of activation of these methods could be interpreted by the user to understand the status of the lens. The user can use various methods to signal the lens that he/she is ready to start or other acknowledgements. For instance, the lens could be opened and inserted into the eyes awaiting a command. Blinking or even closing one's eyes could start the process. The lens then would signal the user that it is starting and then when it finishes. If the lens requires a follow-up, it signals the user and the user signals back with a blink or eye closing. The smart phone may send an instruction to the ophthalmic device to perform a communication operation. For example, if an instruction is received via the app or a system parameter is changed via the app, the smart phone may cause the ophthalmic device to perform a communication operation (e.g., associated with the specific event). If the smart phone is receiving a call, message, or other notification, the smart phone may cause (e.g., by transmitting a signal indicative of the call, message, or notification) the ophthalmic device to perform a communication operation.

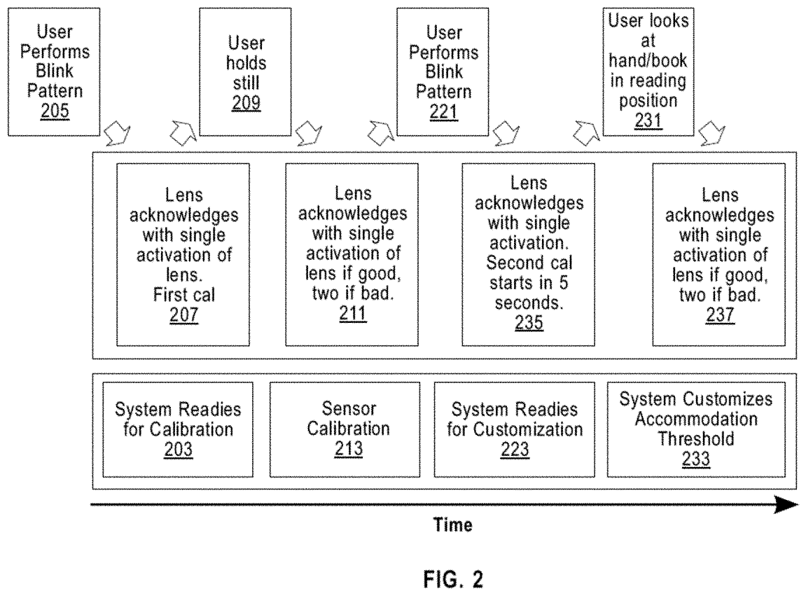

Referring to FIG. 2, one method according to an embodiment of the present invention is depicted. The process starts at an initial time (far left of the figure) and proceeds forward in time. Once the lens (see FIG. 3) are inserted, the system readies for calibration 203. The user performs a blink pattern 205. The lens acknowledges with a single activation of the lens 207 as part of a first calibration. The user holds still 209 as the system and the sensor calibration 213 starts. The lens acknowledges with a single activation of the lens if the first stage of calibration is good 211. If the initial calibration is bad, then the lens acknowledges with a double activation 211. If the calibration is bad, then the user must restart the calibration process 205. After the initial calibration, the system is ready for customization 223. The user conducts another blink pattern 221. The lens acknowledges with a single activation of the lens and a second calibration, customization, is started in some fixed time 235 as part the system customization accommodation threshold 233. The user then looks at either their hand or a book at reading position 231. The lens acknowledges with a single activation of the lens if the second stage of calibration customization is good 237. If the second stage of calibration customization is bad, then the user must restart the calibration customization process 221. Once the lens acknowledges with a single activation of the lens that the second stage of calibration customization is good 237 the system has the completed customization accommodation calibration and the lens are ready for full use by the user.

The communication operation may be performed before, after, or during any of the steps of FIG. 2. For example, the communication operation may be performed as part of the acknowledgment of one or more of steps 207, 211, 235, and 237. It should be understood that the single activation of the lens described above and shown in FIG. 2 is just one example of a communication operation. The acknowledgement may be given to the user via any communication operation described herein, such as a flicker operation. A positive acknowledgement may be given in some situations and a negative acknowledgement may be given in other situations. For example, the negative acknowledgement may comprise a shorter flicker operation than a flicker operation associated with the positive acknowledgment, or vice versa. A pattern of communication operations, such as a pattern of flicker operations, may be communicated when the calibration/customization is completed. For example, two positive acknowledgements (e.g., two flicker operations associated with positive operations) may be communicated to the user.

Other embodiments to customize the threshold can be accomplished. One way is to have the user's doctor determine the comfortable distance for the user by measuring the distance between the eyes of the patient, the typical distance for certain tasks, and then calculate the threshold. From there, using trial and error methods, determine the comfortable distance. Various thresholds can be programmed into the lens and the user can select the task appropriate threshold.

Another method is to allow the user to select his threshold himself. The lens can use the same system that it uses to measure the user's relative eye position to set the accommodation threshold. Where the user's preference of when to activate the extra lens power. There is an overlap where the user's eyes can accommodate unassisted to see adequately and where the user's eyes also can see adequately with the extra power when the lens is active. At what point to activate may be determined by user preference. Providing a means for the user to set this threshold, improves the comfort and utility of the lenses. The procedure follows this sequence:
The user prompts the system to start the sequence. Initially the system could prompt the user as a part of the initial calibration and customization. The user may be prompted by a communication operation associated with starting the sequence;
The lenses are activated. The ability to achieve a comfortable reading position and distance requires the user to actually see the target, thus the lens are in the accommodation state;
The user focuses on a target which is at a representative distance while the system determines the distance based on the angles of the eyes by using the sensor information (accelerometers or magnetometers); after several measurements and noise reduction techniques the system calculates a threshold and indicates that it has finished. The indication may be given to the user by a communication operation as described herein. For example, a flicker operation may be performed;
The new threshold has been determined. A slight offset is subtracted to effectively place the threshold a little farther away, thus creating hysteresis. This is necessary to move the threshold slightly longer (angle slightly lower) in order to guarantee when the user is in the same position, the system will accommodate even with small head or body position differences; The value of this hysteresis could be altered by an algorithm that adapts to user habits. Also, the user could manually change the value if the desired by having the system prompt the user to move the focus target to a position that the user does not want the lenses to activate all the while focusing on the target. The system would deactivate the lenses and then determine this distance. The Hysteresis value is the difference in the deactivate distance and the activate distance. Lenses are now on dependent on the new threshold and hysteresis values.

To have a good user experience, the user needs to have a confirmation that the system has completed any adjustments or customization. In addition, the system needs to determine if the user performed these tasks properly and if not, and then request that the user preforms the procedure again. Such cases include may include excessive movement during measurement, head not straight, lens out of tolerance, etc. The interactive experience will have far fewer frustrated or unhappy users.

Feedback may be given through various means. For example, a communication operation may be performed to indicate feedback to the user. The communication operation may be associated with completion of an adjustment or customization. The communication operation may comprise a pattern of communications, such as multiple flicker operations in succession. A communication operation may comprise transmitting data indicative of a communication to a mobile phone. A phone app may be configured to provide the communication to the user. The ophthalmic device may attempt to send a message to the mobile phone to display information to the user. If the attempt fails, then the ophthalmic device may communicate the information using a communication operation (e.g., flicker operation, activation of lens, and/or the like). The methods as discussed for calibration per the embodiments of the present invention can be done in conjunction with the use of a smartphone app with use of the communication elements as described in reference to FIG. 1 and with reference to FIG. 3 hereafter.

As a part of continual improvement for the lens, data for the lenses may be collected and sent back to the manufacturer (anonymously) via the smartphone app to be used to improve the product. Collected data include, but are not limited to, accommodation cycles, errors, frequency that poor conditions occur, number of hours worn, user set threshold, etc.

Other communication operations and methods to indicate operation include, but not limited to, blinking lights, vibrating haptics drivers, and activating the lens. Various patterns of activation of these methods could be interpreted by the user to understand the status of the lens. For example, a focal length, refractive power, accommodation parameter, and/or the like may be modified to communicate an acknowledgement or other feedback to the user.

Referring now to FIG. 3, shown is another exemplary implementation according to an embodiment of the present invention in which sensing and communication may be used to communicate between a pair of contact lenses (305, 307). Pupils (306, 308) are illustrated for viewing objects. The contact lenses (305, 307) include embedded elements, such as those shown in FIG. 1. The embedded elements (309, 311) included for example 3-axis accelerometers/magnetometers, lens activators, calibration controller, a system controller, memory, power supply, and communication elements as is described in detail subsequently. A communication channel 313 between the two contact lenses (305, 307) allows the embedded elements to conduct calibration between both two contact lenses (305, 307). Communication may also take place with an external device, for example, spectacle glasses, key fob, dedicated interface device, or a smartphone. Communication between the contact lenses (305, 307) is important to detect proper calibration. Communication between the two contact lenses (305, 307) may take the form of absolute or relative position, or may simply be a calibration of one lens to another if there is suspected eye movement. If a given contact lens detects calibration different from the other lens, it may activate a change in stage, for example, switching a variable-focus or variable power optic equipped contact lens to the near distance state to support reading. Other information useful for determining the desire to accommodate (focus near), for example, lid position and ciliary muscle activity, may also be transmitted over the communication channel 313. It should also be appreciated that communication over the channel 313 could comprise other signals sensed, detected, or determined by the embedded elements (309, 311) used for a variety of purposes, including vision correction or vision enhancement.

The communications channel (313) comprises, but not limited to, a set of radio transceivers, optical transceivers, or ultrasonic transceivers that provide the exchange of information between both lens and between the lenses and a device such as a smart phone, FOB, or other device used to send and receive information. The types of information include, but are not limited to, sensor readings showing position, the results of system controller computation, synchronization of threshold and activation. In addition, the device or smart phone could upload settings, sent sequencing signals for the various calibrations, and receive status and error information from the lenses.

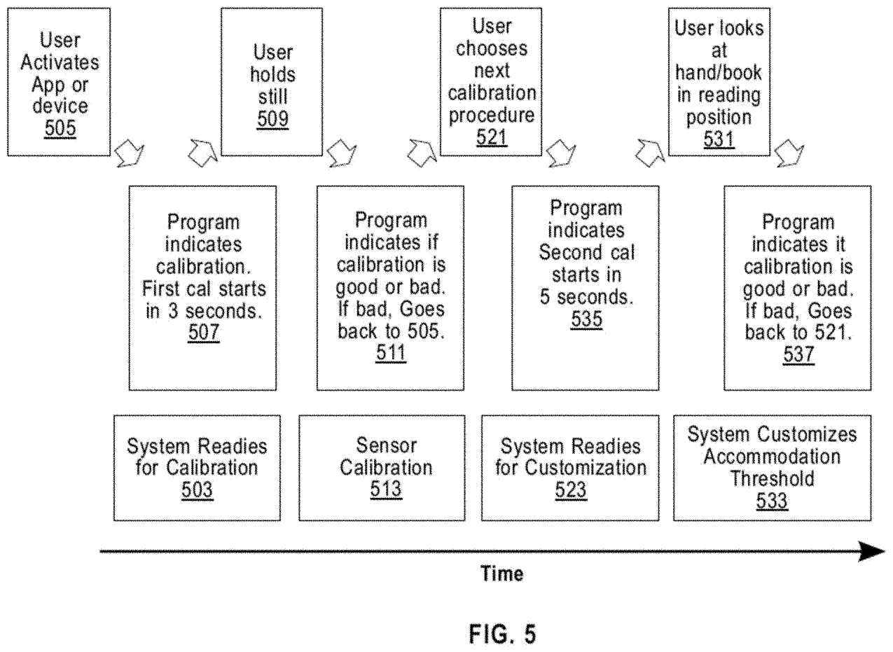

Still referring to FIG. 3, the contact lens (305, 307) further communicates with a smart phone (316) or other external communication device. Specifically, an app 318 on the smart phone (316) communicates to the contact lens (305, 307) via a communication channel (320). The functionally of the app (318) follows the process as outlined with referenced to FIG. 5 (described hereafter) and instructs the user when to perform the required eye movements. In addition, the device or smart phone (316) could upload settings, sent sequencing signals for the various calibrations, and receive status and error information from the contact lenses (305, 307).

The contact lens (e.g., or other ophthalmic lens) may be configured to perform a communication operation together or separately. For example, a communication operation may be associated with a particular eye of the user. Feedback may be given via ophthalmic device disposed in a right eye or a left eye of the user. Negative communication (e.g., negative feedback, such as an operation failed) may be associated with one of the ophthalmic devices (e.g., in one eye). Positive communication (e.g., positive feedback, such as an operation was successful) may be associated with the other one of the ophthalmic devices (e.g., in the other eye). Additionally, a communication operation may comprise performing one communication operation (e.g., flicker operation, activation) in one ophthalmic device while performing another communication operation (e.g., reverse flicker operation, deactivation) in another ophthalmic device. A communication operation may comprise performing one communication operation (e.g., flicker operation, activation, pattern of operations) in one ophthalmic device followed by performing another communication operation (e.g., flicker operation, activation, deactivation, pattern of operations) in another ophthalmic device. Use of both ophthalmic devices may communicate different information than use of one ophthalmic device to perform a communication operation.

Referring to FIG. 5, another method according to an embodiment of the present invention is depicted. The process starts at an initial time (far left of the figure) and proceeds forward in time. Once the lens (see FIG. 3) are inserted, the system readies for calibration 503. User activates App or device 205. The app program indicates calibration and the first calibration starts in 3 seconds 507 as part of a first calibration. The user holds still 509 as the system and the sensor calibration 513 starts. The program indicates if calibration is good or bad 511. If calibration is bad the program restarts and goes back (to step 505) 511. After the initial calibration, the system is ready for customization 523. The user chooses the next calibration procedure 521. The program indicates the second calibration will start in 5 seconds 535 as part the system customization accommodation threshold 533. The user then looks at either their hand or a book at reading position 531. The program determines if second stage of calibration customization is good 537. If the second stage of calibration customization is bad, then the user must restart the calibration customization process 521. Once the program acknowledges that the second stage of calibration customization is good 537 the system has the completed customization accommodation calibration and the lenses are ready for full use by the user.

The communication operation may be performed before, after, or during any of the steps of FIG. 5. For example, the communication operation may be performed as part of the indications of one or more of steps 507, 511, 535, and 537. Thus, a user may receive an indication in both the app and the ophthalmic device. The ophthalmic device may be used to communicate if the app fails to communicate information. The indication may be given to the user via any communication operation described herein, such as a flicker operation. A positive indication may be given in some situations, and a negative indication may be given in other situations. A pattern of communication operations, such as a pattern of flicker operations, may be communicated when the calibration/customization of the accommodation threshold is completed. For example, two positive indications (e.g., two flicker operations associated with positive operations) may be communicated to the user.

Referring to FIG. 6, another method according to an embodiment of the present invention is depicted. At step 602, an occurrence of an event may be determined. The occurrence of the event may be determined by a processor disposed on or in an ophthalmic device. The ophthalmic device may be disposed within or upon an eye of a user. The ophthalmic device may comprise an ophthalmic lens and a variable-optic element. The variable-optic element may be configured to change a parameter of the ophthalmic lens. The ophthalmic lens may have an optic zone and a peripheral zone. The variable-optic element may be incorporated into the optic zone and the processor may be incorporated into the peripheral zone. The ophthalmic lens may comprise a contact lens or an implantable lens, or a combination of both.

The event may comprise completion of a calibration operation, a customization operation, and/or the like. The event may comprise one or more of receipt of a user instruction, modification of a system parameter, receipt of a notification from a remote device, a condition is triggered, and/or the like.

At step 604, the ophthalmic lens may be caused to adjust from a first parameter value to a second parameter value indicative of the occurrence of the event. The first parameter value may be associated with a first focal length of the ophthalmic lens. The second parameter value may be associated with a second focal length of the ophthalmic lens. The first parameter value may be based on a first accommodation setting of the ophthalmic lens. The second parameter value may be based on a second accommodation setting of the ophthalmic lens. Causing the ophthalmic lens to adjust from a first parameter value to a second parameter value may comprise causing the ophthalmic lens to be activated from a deactivated state, causing the ophthalmic lens to be deactivated from an activated state, causing the ophthalmic lens disrupt focus (e.g., cause blurring), and/or the like.

In an aspect, the method 600 may further comprise determining a level of importance associated with the event and determining one or more of the time threshold or the second parameter value based on the level of importance. For example, the second parameter value may be higher for a higher level of importance than for a lower level of importance.

At step 606, it may be determined that a time threshold is satisfied. One or more of the second parameter value or the time threshold may be selected based on an association of the one or more of the second parameter value or the time threshold with a predefined communication.

At step 608, the ophthalmic lens may be caused to adjust the ophthalmic lens from the second parameter value to the first parameter value. The ophthalmic lens may be caused to adjust the ophthalmic lens from the second parameter value to the first parameter value in response to determining that the is satisfied.

As an illustration, the method 600 may be performed as part of a calibration operation. The user may be prompted to perform a calibration operation by the ophthalmic device performing the method 600. For example, the ophthalmic lens may be activated for a second (e.g., or other time period) then deactivated to prompt the user to perform a calibration step, such as looking at one or more distance. The user may look at a first distance, such as at a nearby book, until receiving feedback (e.g., a temporary change of the refractive power) from the ophthalmic device. The user may look at a second distance, such as a distant wall, until receiving feedback (e.g., a temporary change of the parameter value, such as a refractive power) from the ophthalmic device. Then, when the calibration operation is completed, the ophthalmic device may perform the same communication operation (e.g., activation followed by deactivation) or another communication operation, such as a flicker operation, in which the parameter value (e.g., refractive power) of the ophthalmic lens is changed temporarily.

As another illustration, the event may comprise a power level of a battery falling below a threshold. A second parameter value may be determined based on the threshold. For example, if the threshold is a 50 percent power level (e.g., 5 hours of battery left), an importance level may be low or moderate. If the threshold is a 5 percent power level (e.g., 10 minutes of battery left), an importance level may be high. Thus, the second parameter value for a high importance level may provide a greater change from the first parameter value than a second parameter level for a low or moderate importance level. For example, at the 50 percent threshold, one or more regions of the ophthalmic device (e.g., active region, region of the ophthalmic lens) may be temporarily modified to provide a subtle haze or blurred effect. At the 5 percent threshold, one or more regions of the ophthalmic device (e.g., active region, region of the ophthalmic lens) may be temporarily modified to provide a more distracting effect of a prolonged or greater haziness, greater opacity, illumination or darkening of one or more regions (e.g., concentric rings), a greater number of repetitions of a pattern, and/or the like.

As another illustration, the second parameter can relate to a flicker operation. The flicker operation may comprise applying the second parameter value for a time period and then returning to the second parameter. The number of times the flicker operation is performed may be based on the event detected. The speed and/or frequency of performing or repeating the flicker operation may be based on the event detected. If the event has an associated high importance, then the number of times, the speed, and/or the frequency may be increased as compared to an event associated with a lower importance. For example, if a message from a contact (e.g., friend) is received, the ophthalmic device may perform 2 or 3 flicker operations if the friend is assigned as a favorite friend. Messages from other contacts may be communicated to the user using only 1 flicker operation. If the ophthalmic device determines that a user has a high health risk (e.g., based on a measure or determined pulse, dehydration, and/or other health metric) the flicker operation may be performed 10 times. Any pattern may be used as appropriate.

As another illustration, a user may determine one or more communication operations to represent different types of communications. A user interface (e.g., projected by the ophthalmic device, accessed via a mobile device, via a web portal, or a remote device) may allow a user to specify new patterns and/or select predefined patterns. The user may specify which zones are used, opacity levels, number of regions (e.g., number of concentric rings), polarization, eye polarity, speed of transition between the first parameter value and the second parameter value, number of repetitions of the communication operation, a particular sequence of changing parameter values, and/or the like for corresponding communications operations.

As another illustration, communication operations may be selected to help train a user to perform a task. The task may be health related, such as exercising within a heart rate zone, exercise according to a certain pace, standing up a certain amount of time, maintaining an activity level, maintaining posture, maintaining a hydration level, and/or the like. The ophthalmic device may leverage various sensors to determine whether the user is performing the task appropriately (e.g., within a threshold). If the user is not performing the task appropriately, then a communication operation, such as a flicker operation may be performed to notify the user. For example, if a user falls below an exercise pace, activity count (e.g., step count), and/or heart rate, such may be detected as an event, and a corresponding second parameter value may be selected. The task may be related to working, reading, studying, maintaining attention, and/or the like. If a user falls below a reading pace, such may be detected as an event, and a corresponding second parameter value may be selected.

As another illustration, the second parameter value may be one of several second parameter values. For example, causing the ophthalmic lens to adjust from the first parameter value to the second parameter value indicative of the occurrence of the event comprises causing the ophthalmic lens to adjust at least one of an opacity, a polarization, a focal path, an optical power, a focal length, a refractive index, or a gradient of at least a portion of the ophthalmic device. The at least the portion of the ophthalmic device may comprise at least a portion of the ophthalmic lens. The communication operation may comprise modifying parameter values for any combination of the opacity, the polarization, the focal path, the optical power, the focal length, the refractive index, or the gradient. For example, first region (e.g., a first concentric ring) may be modified from a first parameter to a second parameter. For example, opacity may be modified in the first region. A second region (e.g., a second concentric ring, a lenslet, a portion of the ophthalmic lens in the active zone) may be modified from a third parameter value to a fourth parameter value. For example, a focal length of the ophthalmic lens may be modified, a polarization may be adjusted, a refractive index may be changed, and/or the like.

Referring to FIG. 7, another method according to an embodiment of the present invention is depicted. At step 702, data may be received by a processor disposed on or in an ophthalmic device. The ophthalmic device may be disposed within or upon an eye of a user. The ophthalmic device may comprise an ophthalmic lens and a variable-optic element. The variable-optic element may be configured to change a refractive power of the ophthalmic lens. The ophthalmic lens may have an optic zone and a peripheral zone. The variable-optic element may be incorporated into the optic zone. The processor may be incorporated into the peripheral zone. The ophthalmic lens may comprise a contact lens or an implantable lens, or a combination of both.

The data may be indicative of an event. The data may be received from a sensor disposed in the ophthalmic device. The data may be a classification result of data received from the sensor. The data may be received from a function executed by the processor. The data may comprise a result of a logical determination. The data may indicate completion of a calibration operation, such as adjustment of an accommodation threshold. The data may indicate one or more of receipt of a user instruction, modification of a system parameter, or receipt of a notification from a remote device. The data may indicate a condition is triggered. The condition may comprise one or more of a battery level, change in connection status with a device, or change in connection status with a network.

At step 704, modification of an accommodation setting (e.g., or other parameter, calibration setting, vergence setting) may be caused based on the data. The modification may cause the ophthalmic lens to be activated or deactivated. The modification may increase or decrease a focal length of the ophthalmic lens. The modification may decrease or increase one or more of clarity or focus.

At step 706, a return to the prior accommodation setting may be caused after a predefined time period. One or more of the modification or the predefined time period may be selected based on an association of the one or more of the modification or the predefined time period with a predefined communication to the user. The predefined time period may be a fixed time or a time when an event occurs, such as receiving feedback from the user (e.g., a gesture such as moving eyes, blinking, tilting head).

As an illustration, the data may be received from a local or remote device, such as a mobile phone of the user. The data may indicate that the user has received a message or is receiving a call or notification. The ophthalmic device may be configured to change an accommodation parameter, thereby changing a focal length and/or refractive power of the ophthalmic lens. For example, the ophthalmic device may perform a flicker operation. The user's vision may be temporarily blurred during the time period, or the user may be caused to focus at a distance, for a second, a half second, or other time period. The method may be repeated while the call is still ringing the device, or until the user acknowledges (e.g., by performing a gesture, such as blinking of eyes according to a pattern).

Referring to FIG. 8, another method according to an embodiment of the present invention is depicted. At step 802, it may be determined to communicate with a user via the ophthalmic device. The determination may be made by a processor disposed on or in an ophthalmic device. The ophthalmic device may be disposed within or upon an eye of the user. The ophthalmic device may comprise an ophthalmic lens and a variable-optic element. The variable-optic element may be configured to change a refractive power of the ophthalmic lens. The ophthalmic lens may comprise a contact lens or an implantable lens, or a combination of both.

Determining to communicate with the user via the ophthalmic device may comprise determining to communicate completion of a calibration sequence. Determining to communicate with the user via the ophthalmic device may comprise determining to communicate receipt of a user instruction, modification of a system parameter, receipt of a notification from a remote device, or a condition is triggered. Determining to communicate with the user via the ophthalmic device may comprise determining a type of information to communicate. The first accommodation parameter may be determined based on an association with the type of information. The type of information may comprise negative feedback, positive feedback, notification from remote device, completion of an operation (e.g., calibration), warning, and/or the like.

At step 804, a first accommodation parameter may be determined. The first accommodation parameter may be determined based on (e.g., in response to) determining to communicate with the user. Determining the first accommodation parameter may comprise determining a sequence of adjustments associated with communicating with the user. Determining the first accommodation parameter may comprise determining the first accommodation parameter based on an association of the first accommodation parameter with communicating with the user. The first accommodation parameter may comprise a configuration setting of the ophthalmic device that, when used by the ophthalmic device, causes the ophthalmic lens to enable focus at a first distance for the eye.

At step 806, the ophthalmic device may be caused to adjust the ophthalmic lens from a first focal length to a second focal length. The ophthalmic device may be caused to adjust the ophthalmic lens from a first focal length to a second focal length based on the first accommodation parameter.

Causing the ophthalmic device to adjust the ophthalmic lens from the first focal length to the second focal length may comprise causing the ophthalmic lens to activate based on the first accommodation parameter. Causing the ophthalmic device to adjust the ophthalmic lens from the first focal length to the second focal length may comprise causing the ophthalmic lens to adjust focus from a first distance to a second distance.

At step 808, the ophthalmic device may be caused to adjust (e.g., based on a second accommodation parameter) the ophthalmic lens from the second focal length to the first focal length. The ophthalmic device may be caused to adjust the ophthalmic lens from the second focal length to the first focal length in response to a time threshold being satisfied. Causing the ophthalmic device to adjust the ophthalmic lens from the second focal length to the first focal length may comprise causing deactivation of the ophthalmic lens.

The method 800 may comprise determining the time threshold. The time threshold may be determined such that causing the ophthalmic device to adjust the ophthalmic lens provides less than a threshold amount of disruption to the user. The method 800 may further comprise determining that the time threshold is satisfied. Determine that the threshold is satisfied may comprise starting a timer in response to causing, based on the first accommodation parameter, the ophthalmic device to adjust the ophthalmic lens from the first focal length to the second focal length. Determine that the threshold is satisfied may comprise determining that a value of the timer meets or exceeds a value of the time threshold.

As an illustration, a determination may be made to communicate with a user in response to customizing an accommodation threshold. The user may set a custom accommodation threshold. The ophthalmic device may determine to communicate with the user based on the accommodation threshold being changed. Changing of the accommodation threshold by the user may be associated with a sequence of two flicker operations. A user may be wearing to ophthalmic devices. One of the ophthalmic devices may instruct the other ophthalmic device to perform the sequence of communication operations. Timing information and a refractive power setting may be sent as part of the instruction. Both of the ophthalmic devices may perform the flicker operation in parallel (e.g., simultaneously, at the same time).

Referring now to FIG. 9, there is illustrated, in planar view, a wearable electronic ophthalmic device comprising a sensor in accordance with the present disclosure. The ophthalmic device 900 comprises an optic zone 902 and a peripheral zone 904. The optic zone 902 may function to provide one or more of vision correction, vision enhancement, other vision-related functionality, mechanical support, or even a void to permit clear vision. In accordance with the present disclosure, the optic zone 902 may comprise a variable optic element configured to provide enhanced vision at near and distant ranges based on signals sensed from the ciliary muscle. The variable-optic element may comprise any suitable device for changing the focal length of the lens or the refractive power of the lens based upon activation signals from the sensing system described herein. For example, the variable optic element may be as simple as a piece of optical grade plastic incorporated into the lens with the ability to have its spherical curvature changed. The peripheral zone 904 comprises one or more of electrical circuits 906, a power source 908, electrical interconnects 910, mechanical support, as well as other functional elements.

The electrical circuits 906 may comprise one or more integrated circuit die, printed electronic circuits, electrical interconnects, and/or any other suitable devices, including the sensing circuitry described herein. The power source 908 may comprise one or more of battery, energy harvesting, and or any other suitable energy storage or generation devices. It is readily apparent to the skilled artisan that FIG. 9 only represents one exemplary embodiment of an electronic ophthalmic lens and other geometrical arrangements beyond those illustrated may be utilized to optimize area, volume, functionality, runtime, shelf life as well as other design parameters. It is important to note that with any type of variable optic, the fail-safe is distance vision. For example, if power were to be lost or if the electronics fail, the wearer is left with an optic that allows for distance vision.

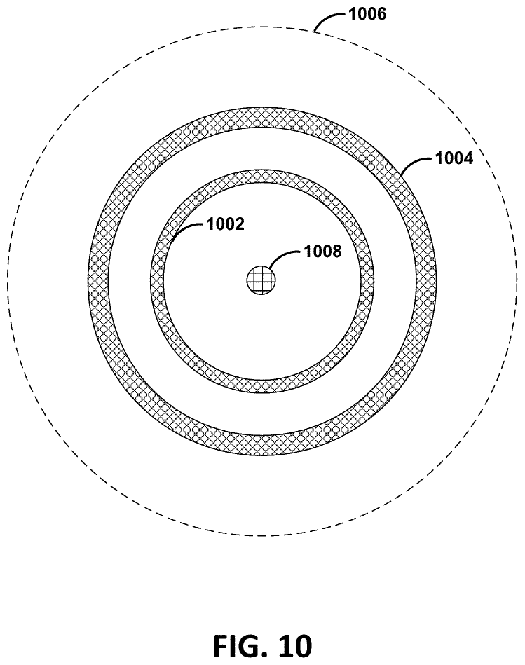

FIG. 10 is a diagram illustration regions of an ophthalmic device. In an aspect, an ophthalmic device may comprise a plurality of regions, such as a first region 1002 and a second region 1004. The first region 1002 and the second region 1004 may comprise concentric rings. The first region 1002 may be surrounded by the second region 1004. The first region 1002 and the second region may be within a zone 1006. The zone 1006 may comprise an active zone (e.g., or optical zone) configured to allow light to pass through to a pupil of the eye. An example lenslet 1008 is also shown. The lenslet 1008 may comprise a liquid meniscus lenslet comprise one or more liquids. The first region 1002, the second region 1004, and the lenslet 1008 may be translucent and/or invisible to the eye of the user while in a first state (e.g., associated with a first parameter value). The state of each of the first region 1002, the second region 1004, and the lenslet 1008 may be modified independently to perform a variety of communication operations as described herein. Though not shown, it is understood, that the first region 1002, the second region 1004, and the lenslet 1008 may be disposed outside the zone 1006 in a peripheral zone. It is also understood that wires may be disposed in positions to produce a current, voltage, and/or electrical field in one or more of the first region 1002, the second region 1004, and the lenslet 1008. The first region 1002, the second region 1004, and the lenslet 1008 may be modified to any of a plurality of states depending on the amount of energy and/or signal provided. The states may change opacity, focal length, focal path, polarization, refractive power, and/or the like to create visual effects that can be perceived by the user to communicate with the user.

It is important to note that the above described elements may be realized in hardware, in software or in a combination of hardware and software. In addition, the communication channel may comprise any include various forms of wireless communications. The wireless communication channel may be configured for high frequency electromagnetic signals, low frequency electromagnetic signals, visible light signals, infrared light signals, and ultrasonic modulated signals. The wireless channel may further be used to supply power to the internal embedded power source acting as rechargeable power means.

The present invention may be a system, a method, and/or a computer program product. The computer program product being used by a controller for causing the controller to carry out aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiments were chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. An ophthalmic system comprising:
an ophthalmic device configured to be disposed within or upon an eye of a user and comprising an ophthalmic lens and a variable-optic element, the variable-optic element being configured to change a parameter of the ophthalmic lens; and
a processor disposed in or on the ophthalmic device, wherein the processor is configured for:
determining occurrence of an event;
causing the ophthalmic lens to adjust from a first parameter value to a second parameter value indicative of the occurrence of the event;
determining that a time threshold is satisfied; and
causing, in response to determining that the time threshold is satisfied, the ophthalmic lens to adjust the ophthalmic lens from the second parameter value to the first parameter value.

2. The ophthalmic system of claim 1, wherein causing the ophthalmic lens to adjust from the first parameter value to the second parameter value indicative of the occurrence of the event comprises causing the ophthalmic lens to adjust at least one of an opacity, a polarization, a focal path, an optical power, a focal length, a refractive index, or a gradient of at least a portion of the ophthalmic device.

3. The ophthalmic system of claim 2, wherein the at least the portion of the ophthalmic device comprises at least a portion of the ophthalmic lens.

4. The ophthalmic system of claim 1, further comprising a determining a level of importance associated with the event and determining one or more of the time threshold or the second parameter value based on the level of importance.

5. The ophthalmic system of claim 1, wherein the ophthalmic lens has an optic zone and a peripheral zone, and wherein the variable-optic element incorporated into the optic zone and the processor is incorporated into the peripheral zone.

6. The ophthalmic system of claim 1, wherein the event comprises completion of a calibration operation.

7. The ophthalmic system of claim 1, wherein the event comprises one or more of receipt of a user instruction, modification of a system parameter, receipt of a notification from a remote device, or a condition is triggered.

8. The ophthalmic system of claim 1, wherein the first parameter value is associated with a first focal length of the ophthalmic lens and the second parameter value is associated with a second focal length of the ophthalmic lens.

9. The ophthalmic system of claim 1, wherein the first parameter value is based on a first accommodation setting of the ophthalmic lens and the second parameter value is based on a second accommodation setting of the ophthalmic lens.

10. The ophthalmic system of claim 1, wherein one or more of the second parameter value or the time threshold are selected based on an association of the one or more of the second parameter value or the time threshold with a predefined communication.

11. The ophthalmic system of claim 1, wherein the ophthalmic lens comprises a contact lens or an implantable lens, or a combination of both.

12. The ophthalmic system of claim 1, wherein causing the ophthalmic lens to adjust from the first parameter value to the second parameter value comprises causing the ophthalmic lens to be activated from a deactivated state.

13. An ophthalmic system comprising:
an ophthalmic device configured to be disposed within or upon an eye of a user and comprising an ophthalmic lens and a variable-optic element, the variable-optic element being configured to change a refractive power of the ophthalmic lens; and
a processor disposed in or on the ophthalmic device, wherein the processor is configured for:
receiving data;
causing modification of an accommodation setting based on the data; and
causing a return to the accommodation setting after a predefined time period.

14. The ophthalmic system of claim 13, wherein the ophthalmic lens has an optic zone and a peripheral zone, and wherein the variable-optic element incorporated into the optic zone and the processor is incorporated into the peripheral zone.

15. The ophthalmic system of claim 13, wherein the data indicates completion of a calibration operation.

16. The ophthalmic system of claim 13, wherein the data indicates one or more of receipt of a user instruction, modification of a system parameter, or receipt of a notification from a remote device.

17. The ophthalmic system of claim 13, wherein the data indicates a condition is triggered, wherein the condition comprises one or more of a batter level, change in connection status with a device, or change in connection status with a network.

18. The ophthalmic system of claim 13, wherein one or more of the modification or the predefined time period are selected based on an association of the one or more of the modification or the predefined time period with a predefined communication to the user.

19. The ophthalmic system of claim 13, wherein the ophthalmic lens comprises a contact lens or an implantable lens, or a combination of both.

20. The ophthalmic system of claim 13, wherein the modification causes the ophthalmic lens to be activated or deactivated.

21. The ophthalmic system of claim 13, wherein the modification increases or decreases a focal length of the ophthalmic lens.

22. The ophthalmic system of claim 13, wherein the modification decreases or increases one or more of clarity or focus.

23. An ophthalmic system comprising:
an ophthalmic device configured to be disposed within or upon an eye of a user and comprising an ophthalmic lens and a variable-optic element, the variable-optic element being configured to change a refractive power of the ophthalmic lens; and
a processor disposed in or on the ophthalmic device, wherein the processor is configured for:
determining to communicate with the user via the ophthalmic device;
determining, based on determining to communicate with the user, a first accommodation parameter;
causing, based on the first accommodation parameter, the ophthalmic device to adjust the ophthalmic lens from a first focal length to a second focal length; and
causing, in response to a time threshold being satisfied, the ophthalmic device to adjust, based on a second accommodation parameter, the ophthalmic lens from the second focal length to the first focal length.

24. The ophthalmic system of claim 23, wherein the processor is further configured for determining the time threshold, wherein the time threshold is determined such that causing the ophthalmic device to adjust the ophthalmic lens provides less than a threshold amount of disruption to the user.

25. The ophthalmic system of claim 23, wherein determining the first accommodation parameter comprises determining a sequence of adjustments associated with communicating with the user.

26. The ophthalmic system of claim 23, wherein determining to communicate with the user via the ophthalmic device comprises determining to communicate completion of a calibration sequence.

27. The ophthalmic system of claim 23, wherein determining to communicate with the user via the ophthalmic device comprises determining to communicate receipt of a user instruction, modification of a system parameter, receipt of a notification from a remote device, or a condition is triggered.

28. The ophthalmic system of claim 23, wherein determining the first accommodation parameter comprises determining the first accommodation parameter based on an association of the first accommodation parameter with communicating with the user.

29. The ophthalmic system of claim 23, wherein the processor is further configured for determining that the time threshold is satisfied by:
starting a timer in response to causing, based on the first accommodation parameter, the ophthalmic device to adjust the ophthalmic lens from the first focal length to the second focal length; and
determining that a value of the timer meets or exceeds a value of the time threshold.

30. The ophthalmic system of claim 23, wherein determining to communicate with the user via the ophthalmic device comprises determining a type of information to communicate, wherein the first accommodation parameter is determined based on an association with the type of information.

31. The ophthalmic system of claim 23, wherein causing, based on the first accommodation parameter, the ophthalmic device to adjust the ophthalmic lens from the first focal length to the second focal length comprises causing the ophthalmic lens to activate based on the first accommodation parameter.

32. The ophthalmic system of claim 23, wherein causing, based on the first accommodation parameter, the ophthalmic device to adjust the ophthalmic lens from the first focal length to the second focal length comprises causing the ophthalmic lens to adjust focus from a first distance to a second distance.

33. The ophthalmic system of claim 23, wherein first accommodation parameter comprises a configuration setting of the ophthalmic device that, when used by the ophthalmic device, causes the ophthalmic lens to enable focus at a first distance for the eye.

34. The ophthalmic system of claim 23, wherein causing the ophthalmic device to adjust, based on a second accommodation parameter, the ophthalmic lens from the second focal length to the first focal length comprises causing deactivation of the ophthalmic lens.

35. The ophthalmic system of claim 23, wherein the ophthalmic lens comprises a contact lens or an implantable lens, or a combination of both.

36. A method comprising:
determining, by a processor disposed on or in an ophthalmic device, an occurrence of an event, wherein the ophthalmic device is disposed within or upon an eye of a user and comprises an ophthalmic lens and a variable-optic element, and wherein the variable-optic element is configured to change a parameter of the ophthalmic lens;
causing the ophthalmic lens to adjust from a first parameter value to a second parameter value indicative of the occurrence of the event;
determining that a time threshold is satisfied; and
causing, in response to determining that the time threshold is satisfied, the ophthalmic lens to adjust the ophthalmic lens from the second parameter value to the first parameter value.

37. The method of claim 36, wherein causing the ophthalmic lens to adjust from the first parameter value to the second parameter value indicative of the occurrence of the event comprises causing the ophthalmic lens to adjust at least one of an opacity, a polarization, a focal path, an optical power, a focal length, a refractive index, or a gradient of at least a portion of the ophthalmic lens.

38. The method of claim 37, wherein the at least the portion of the ophthalmic device comprises at least a portion of the ophthalmic lens.

39. The method of claim 36, further comprising a determining a level of importance associated with the event and determining one or more of the time threshold or the second parameter value based on the level of importance.

40. The method of claim 36, wherein the ophthalmic lens has an optic zone and a peripheral zone, and wherein the variable-optic element incorporated into the optic zone and the processor is incorporated into the peripheral zone.

41. The method of claim 36, wherein the event comprises completion of a calibration operation.

42. The method of claim 36, wherein the event comprises one or more of receipt of a user instruction, modification of a system parameter, receipt of a notification from a remote device, or a condition is triggered.

43. The method of claim 36, wherein the first parameter value is associated with a first focal length of the ophthalmic lens and the second parameter value is associated with a second focal length of the ophthalmic lens.

44. The method of claim 36, wherein the first parameter value is based on a first accommodation setting of the ophthalmic lens and the second parameter value is based on a second accommodation setting of the ophthalmic lens.

45. The method of claim 36, wherein one or more of the second parameter value or the time threshold are selected based on an association of the one or more of the second parameter value or the time threshold with a predefined communication.

46. The method of claim 36, wherein the ophthalmic lens comprises a contact lens or an implantable lens, or a combination of both.

47. The method of claim 36, wherein causing the ophthalmic lens to adjust from a first parameter value to a second parameter value comprises causing the ophthalmic lens to be activated from a deactivated state.

48. A method comprising:
receiving, by a processor disposed on or in an ophthalmic device, data, wherein the ophthalmic device is disposed within or upon an eye of a user and comprises an ophthalmic lens and a variable-optic element, and wherein the variable-optic element is configured to change a refractive power of the ophthalmic lens;
causing modification of an accommodation setting based on the data; and
causing a return to the accommodation setting after a predefined time period.

49. The method of claim 48, wherein the ophthalmic lens has an optic zone and a peripheral zone, and wherein the variable-optic element incorporated into the optic zone and the processor is incorporated into the peripheral zone.

50. The method of claim 48, wherein the data indicates completion of a calibration operation.

51. The method of claim 48, wherein the data indicates one or more of receipt of a user instruction, modification of a system parameter, or receipt of a notification from a remote device.

52. The method of claim 48, wherein the data indicates a condition is triggered, wherein the condition comprises one or more of a batter level, change in connection status with a device, or change in connection status with a network.

53. The method of claim 48, wherein one or more of the modification or the predefined time period are selected based on an association of the one or more of the modification or the predefined time period with a predefined communication to the user.

54. The method of claim 48, wherein the ophthalmic lens comprises a contact lens or an implantable lens, or a combination of both.

55. The method of claim 48, wherein the modification causes the ophthalmic lens to be activated or deactivated.

56. The method of claim 48, wherein the modification increases or decreases a focal length of the ophthalmic lens.

57. The method of claim 48, wherein the modification decreases or increases one or more of clarity or focus.

58. A method comprising:
determining, by a processor disposed on or in an ophthalmic device, to communicate with a user via the ophthalmic device, wherein the ophthalmic device is disposed within or upon an eye of the user and comprises an ophthalmic lens and a variable-optic element, and wherein the variable-optic element is configured to change a refractive power of the ophthalmic lens;
determining, based on determining to communicate with the user, a first accommodation parameter;
causing, based on the first accommodation parameter, the ophthalmic device to adjust the ophthalmic lens from a first focal length to a second focal length; and
causing, in response to a time threshold being satisfied, the ophthalmic device to adjust, based on a second accommodation parameter, the ophthalmic lens from the second focal length to the first focal length.

59. The method of claim 58, further comprising determining the time threshold, wherein the time threshold is determined such that causing the ophthalmic device to adjust the ophthalmic lens provides less than a threshold amount of disruption to the user.

60. The method of claim 58, wherein determining the first accommodation parameter comprises determining a sequence of adjustments associated with communicating with the user.

61. The method of claim 58, wherein determining to communicate with the user via the ophthalmic device comprises determining to communicate completion of a calibration sequence.

62. The method of claim 58, wherein determining to communicate with the user via the ophthalmic device comprises determining to communicate receipt of a user instruction, modification of a system parameter, receipt of a notification from a remote device, or a condition is triggered.

63. The method of claim 58, wherein determining the first accommodation parameter comprises determining the first accommodation parameter based on an association of the first accommodation parameter with communicating with the user.

64. The method of claim 58, further comprising determining that the time threshold is satisfied by:
starting a timer in response to causing, based on the first accommodation parameter, the ophthalmic device to adjust the ophthalmic lens from the first focal length to the second focal length; and
determining that a value of the timer meets or exceeds a value of the time threshold.

65. The method of claim 58, wherein determining to communicate with the user via the ophthalmic device comprises determining a type of information to communicate, wherein the first accommodation parameter is determined based on an association with the type of information.

66. The method of claim 58, wherein causing, based on the first accommodation parameter, the ophthalmic device to adjust the ophthalmic lens from the first focal length to the second focal length comprises causing the ophthalmic lens to activate based on the first accommodation parameter.

67. The method of claim 58, wherein causing, based on the first accommodation parameter, the ophthalmic device to adjust the ophthalmic lens from the first focal length to the second focal length comprises causing the ophthalmic lens to adjust focus from a first distance to a second distance.

68. The method of claim 58, wherein first accommodation parameter comprises a configuration setting of the ophthalmic device that, when used by the ophthalmic device, causes the ophthalmic lens to enable focus at a first distance by the eye.

69. The method of claim 58, wherein causing the ophthalmic device to adjust, based on a second accommodation parameter, the ophthalmic lens from the second focal length to the first focal length comprises causing deactivation of the ophthalmic lens.

70. The method of claim 58, wherein the ophthalmic lens comprises a contact lens or an implantable lens, or a combination of both.
